(19) United States
(12) Patent Application Publication
(10) Pub. No.: US 2007/0083053 A1
(43) Pub. Date: Apr. 12, 2007

(54) Process for producing indole compound

(75) Inventors: Yasuhiro Sakurai, Funabashi-shi (JP); Tomohisa Utsunomiya, Funabashi-shi (JP); Norio Tanaka, Funabashi-shi (JP)

Correspondence Address: OLIFF & BERRIDGE, PLC, P.O. BOX 19928, ALEXANDRIA, VA, 22320, US

(73) Assignee: Nissan Chemical Industries, LTD., Tokyo (JP)

(21) Appl. No.: 10/562,115

(22) PCT Filed: Jun. 25, 2004
(86) PCT No.: PCT/JP04/09001

§ 371 Date: Jan. 19, 2006

(30) Foreign Application Priority Data
Jun. 27, 2003 (JP) 2003-184359

(51) Int. Cl. C07D 209/04

(52) U.S. Cl. 548/508

(57) ABSTRACT

There is provided a novel process for producing an indole derivative which comprises cyclizing 2-nitrobenzylcarbony compound in the presence of a catalyst comprising a Group VIII metal of the Periodic Table, characterized by conducting the cyclization in a gas atmosphere containing carbon monoxide. The process enables an indole compound to be selectively produced in a high yield from 2-nitrobenzylcarbonyl compound, and hardly yields an indoline compound as a reduction by-product that has been a problem in the catalytic hydrogenation method employing a noble metal catalyst. The indole derivative produced by the present process is useful for various fine chemical intermediates including compounds and physiologically active substances such as pharmaceuticals and agrochemicals.

### TECHNICAL FIELD

The present invention relates to a process for producing indole compound useful as several fine chemical intermediates represented by physiologically active substances such as pharmaceuticals and agrochemicals, etc.

### BACKGROUND ART

As a process for producing indole compound, an example in N-o-tolyl-acetamide is reacted with barium oxide at 360° C. to obtain 2-methyl indole is known (Patent Document 1). Similarly, there are also examples in which sodium amide (Non-patent Document 1) or sodium methoxide (Non-patent Document 2) is used, but these examples require a high temperature and produce a large amount of by-product and the yield is not so high.

Although there is an example in which a phenyl hydrazone of acetone is reacted with sodium hydroxide at 240° C. to obtain 2-methyl indole, it produces a large amount of by-product and has a low yield (Non-patent Document 3). Further, although it is known to produce 2-methyl indole by reacting 2-nitro-1-(2-nitrophenyl) propene with hydrogen in the presence of 10% palladium catalyst supported on active carbon, the yield is 81% (Non-patent document 4). In addition, although it is reported to obtain 2-methyl indole in a yield of 64% by reacting aniline with tris(2-hydroxypropyl) amine hydrochloride in the presence of tin dichloride, ruthenium trichloride and triphenyl phosphine at 180° C., the yield is low (Non-patent Document 5).

The process for producing indole compound from 2-nitrobenzylcarbonyl compound includes for example, a report in which 2-nitrophenyl acetone is reduced with iron in the presence of acetic acid and sodium acetate to obtain 2-methyl indole in a yield of 68% (Non-patent Document 6), a report in which 4-fluoro-2-nitrophenyl acetone is reacted with zinc in an acetic acid aqueous solution to obtain 6-fluoro-2-methyl indole in a yield of 95% (Patent Document 2), and the like. However, these processes discharge a large amount of iron oxide or zinc oxide as waste in post-treatment, and influence adverse effect on environment. In addition, although the latter states that a catalytic reduction in the presence of a catalyst such as palladium, Raney nickel, platinum or the like also provides similar products, it does not disclose any working examples corresponding thereto.

Although reports in which 2-nitrostyrenes as a starting material are reductively cyclized with carbon monoxide to obtain corresponding indole compounds are found here and there (Non-patent Documents 7 and 8), one of these documents uses a selenium catalyst that is a special catalyst and lacks practicality, and the reaction in the other document smoothly proceeds when a special catalyst system being Pd(TMB)₂TMPhen wherein TMB is 2,4,6-trimethylbenzoic anion and TMPhen is 3,4,7,8-tetramethyl-1,10-phenathrorine is utilized, but if a severe condition being a reaction temperature of 180° C. and carbon monoxide pressure of 60 atm is not applied, the formation of by-product by dimerization at α-position of styrene is unavoidable and the yield is lowered. Further, it is known also a process for producing indole in which o-nitrostyrene compound is cyclized in the presence of carbon monoxide under a reducing condition (Patent Document 3).

On the other hand, conventionally, as important intermediates for fine chemical production, several benzylcarbonyl compounds and the production processes thereof have been developed. As 2-nitrobenzyl carbonyl compounds have two kinds of functional groups being nitro group and carbonyl group in the molecule, they are important intermediates for the production of several heterocyclic compounds. In particular, several indole compounds induced from this compound group have been synthesized with a central focus on physiologically active substances through the ages.

The utility of the compound group as fungicide by use of the indole compounds obtained as mentioned above is also known (Patent Document 4). In particular, it is studied a production process by use of 6-fluoro-2-methylindole that is an indole compound having a high utility among the compounds and that uses as an important intermediate 3-(4-fluoro-2-nitrophenyl) acetone that can be relatively easily synthesized. However, when 4-fluoro-2-nitrophenylacetone is actually reduced with hydrogen gas in the presence of active carbon-supported palladium catalyst, 6-fluoro-2-methylindoline is formed as by-product, and thus the yield of 6-fluoro-2-methylindole is about 70%. This is because 1-hydroxy-2-methylindole is formed as a reaction intermediate in a relation of tautomerism with 2-methylindolenine N-oxide, and the 2-methylindolenine N-oxide is further reduced to cause 6-fluoro-2-methylindoline. In the reductive cyclization by use of 2-nitrobenzylcarbonyl compound with a noble metal catalyst through catalytic hydrogenation, it is generally difficult to avoid the above-mentioned side reaction.

In addition, in the catalytic hydrogenation reductive reaction by use of any noble metal-supported catalyst, when 2-nitrobenzylcarbonyl compound has any substituent for example halogen atom such as chlorine, bromine, etc. or benzyloxy on the benzene ring, the elimination or break of the substituent easily occurs and thus it is extremely difficult to make only the aimed reductive cyclization reaction preferentially proceed. Therefore, it is desired a process for producing 2-substituted indole compound that uses 2-nitrobenzylcarbonyl compound as a starting material and that is simple and excellent in yield, selectivity and general applicability.
Patent Document 1: DE Patent No. 262327 Specification
Patent Document 2: JP-A-47-38963 (1972)
Patent Document 3: International Patent Publication WO02/48104 pamphlet
Patent Document 4: International Patent Publication WO99/21851 pamphlet
Non-patent Document 1: Bull. Soc. Chim. Fr., 4, 1039 (1924)
Non-patent Document 2: Org. Syn., 27, 94 (1942)
Non-patent Document 3: Chem. Ber. 81, 266, 270 (1948)
Non-patent Document 4: Heterocycles, 55, 95 (2001)
Non-patent Document 5: Tetrahedron, 3321 (2001)
Non-patent Document 6: J. Org. Chem., 48, 2066 (1983)
Non-patent Document 7: Tetrahedron Lett., 40, 5717 (1999)
Non-patent Document 8: J. Molecular Catalysis, 87, 203 (1994)

### DISCLOSURE OF THE INVENTION

### PROBLEM TO BE SOLVED BY THE INVENTION

The problem to be solved by the invention is to provide a process for producing indole compound that is industrially advantageous and novel, and has a high general applicability.

### MEANS FOR SOLVING THE PROBLEM

The present inventors eagerly investigated in order to solve the above-mentioned problem. As a result of it, they found that indole compounds are obtained in a selective manner and a good yield by using carbon monoxide not a hydrogen-donor in the reduction of 2-nitrobenzylcarbonyl compound in the presence of a metal catalyst, and they completed the present invention.

That is, the present invention relates to the following [1] to [8]:

[1] A process for producing an indole compound of formula (2) 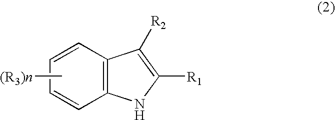
wherein R₁ and R₂ are independently of each other hydrogen atom, an optionally substituted alkyl group, a phenyl group, an alkoxycarbonyl group or an acyl group, R₃ is an optionally substituted alkyl group, a phenyl group, an alkoxy group, a benzyloxy group, an alkoxycarbonyl group, a nitro group or a halogen atom, and n is an integer of 0 to 4, characterized by using carbon monoxide when 2-nitrobenzylcarbony compound of formula (1) 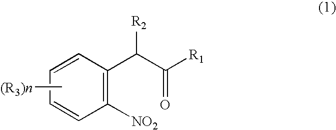
wherein R₁, R₂, R₃ and n have the same meaning as the above, is reduced in the presence of a catalyst comprising a Group VII metal of the Periodic Table.

[2] The process for producing an indole compound as set forth in [1], wherein the catalyst comprising a Group Vil metal of the Periodic Table is a metal catalyst selected from an iron catalyst, a ruthenium catalyst, a palladium catalyst, a cobalt catalyst, a rhodium catalyst, a nickel catalyst and a platinum catalyst.

[3] The process for producing an indole compound as set forth in [1], wherein the catalyst comprising a Group Vil metal of the Periodic Table is a metal catalyst selected from an iron catalyst, a ruthenium catalyst, a palladium catalyst and a platinum catalyst.

[4] The process for producing an indole compound as set forth in [1], wherein the catalyst comprising a Group Vil metal of the Periodic Table is an iron or ruthenium complex catalyst in which carbon monoxide is coordinated.

[5] The process for producing an indole compound as set forth in [1], wherein the catalyst comprising a Group Vil metal of the Periodic Table is a palladiurn catalyst or platinum catalyst in which phosphine type ligand is coordinated.

[6] The process for producing an indole compound as set forth in [1], [2], [3], [4] or [5], wherein R₁ and R₂ are independently of each other hydrogen atom, an optionally substituted alkyl group, an alkoxycarbonyl group or an acyl group, R₃ is an optionally substituted alkyl group or a halogen atom, and n is an integer of 0 to 4.

[7] The process for producing an indole compound as set forth in [1], [2], [3], [4] or [5], wherein R₁ is methyl group, R₂ is hydrogen atom, an alkoxycarbonyl group or an acyl group, R₃ is a halogen atom, and n is an integer of 0 or 1.

[8] The process for producing an indole compound as set forth in [1], [2], [3], [4] or [5], wherein R₁ is methyl group, R₂ is hydrogen atom, R₃ is fluorine atom, and n is an integer of 0 or 1.

### EFFECT OF THE INVENTION

The process according to the present invention produces little indoline compounds being reduction by-products that have been a problem in the prior catalytic hydrogenation method by use of noble metal catalyst, and can produce indole compounds in a selective manner and a high yield from 2-nitrobenzyl carbonyl compounds. In addition, the present invention causes no elimination of the halogen atom on the aromatic ring that has been often a problem in the catalytic hydrogenation method, therefore it is a process for producing indole compound that has a high generality to several substrates.

### BEST MODE FOR CARRYING OUT THE INVENTION

As the compounds to which the present invention is applied, 2-nitrobenzylcarbonyl compounds of formula (1) and indole compounds of formula (2) include compounds wherein R₁ and R₂ are independently of each other hydrogen atom, an optionally substituted alkyl group, a phenyl group, an alkoxycarbonyl group or an acyl group, R₃ is an optionally substituted alkyl group, a phenyl group, an alkoxy group, a benzyloxy group, an alkoxycarbonyl group, a nitro group or a halogen atom, and n is an integer of 0 to 4, preferably compounds wherein R₁ and R₂ are independently of each other hydrogen atom, an optionally substituted alkyl group, an alkoxycarbonyl group or an acyl group, R₃ is an optionally substituted alkyl group or a halogen atom, and n is an integer of 0 to 4, more preferably compounds wherein R₁ is methyl group, R₂ is hydrogen atom, an alkoxycarbonyl group or an acyl group, R₃ is a halogen atom, and n is an integer of 0 or 1, and further preferably compounds wherein R₁ is methyl group, R₂ is hydrogen atom, R₃ is fluorine atom, and n is an integer of 0 or 1.

The 2-nitrobenzylcarbonyl compounds of formula (1) being a starting material of the present invention is produced by any known methods. For example, the compounds include 2-nitrophenylacetone (Tetrahedron Lett., 42,1387 (2001)), 4-chloro-2-nitrophenylacetone (Chem. Pharm. Bull., 17, 605 (1969), and 4-fluoro-2-nitrophenylacetone (JP-A47-38947 (1972)).

Agents and reaction condition used in reducing the 2-nitrobenzylcarbonyl compounds are as follows, but the present invention is not limited thereto.

As the catalyst comprising a Group VIII metal of the Periodic Table, metal catalysts such as an iron catalyst, a ruthenium catalyst, a palladium catalyst, a cobalt catalyst, a rhodium catalyst, a nickel catalyst, a platinum catalyst and the like are preferable, and they can be used in a homogeneous or heterogeneous system.

Examples of the catalysts that can be used in the present reaction are as follows.

The iron catalysts include complex catalysts such as pentacarbonyliron, tetracarbonyl(triphenylphosphine)iron, tricarbonylbis(triphenylphosphine)iron, tetracarbonyl(tricyclohexylphosphine)iron, tetracarbonyl(tributylphosphine)iron, tetracarbonyl(tristolylphosphine)iron, sodium tetracarbonylferrate, bis(triphenylphosphoranediyl)ammonium tetracarbonylhydrideferrate, potassium tetracarbonyl(trimethylsilyl)ferrate, bis(triphenylphosphoranediyl)ammonium tetracarbonyl(trimethylsilyl)ferrate, tetracarbonyl(methylacrylate)iron, tetracarbonyl(ethylacrylate)iron, tetracarbonyl(butylacrylate)iron, tetracarbonyl(methylmethacrylate)iron, tetracarbonyl(ethylmethacrylate)iron, tetracarbonyl(maleic anhydride)iron, tetracarbonyl(maleic acid)iron, tetracarbonyl(fumaric acid)iron, tetracarbonyl(dimethylfumarate)iron, tetracarbonyl(methylcinnamate)iron, tetracarbonyl(cinnamaldehyde)iron, tetracarbonyl(methylisonitrile)iron, tetracarbonyl(ethylisonitrile)iron, tetracarbonyl(butylisonitrile)iron, tricarbonylbis(tricyclohexylphosphine)iron, tricarbonylbis(tributylphosphine)iron, tricarbonylbis(triethylphosphine)iron, tricarbonylbis(tristolylphosphine)iron, bis(triphenylphosphoranediyl)ammonium tricarbonyl(nitrosyl)ferrate, potassium tricarbonyl(triphenylphosphine)ferrate, tetraethylammonium tricarbonylhydride(triphenylphosphine)ferrate, tetrabutylammonium tricarbonylhydride(triphenylphosphine)ferrate, benzyltriethylammonium tricarbonylhydride(triphenylphosphine)ferrate, tetramethylammonium tricarbonylhydride(triphenylphosphine)ferrate, tricarbonyl(1,3-cyclohexadiene)iron, tricarbonyl(1,3-butadiene)iron, tricarbonyl(norbornadiene)iron, tricarbonyl(cyclooctatetraene)iron, tricarbonyl(cyclobutadiene)iron, bromotricarbonyl(allyl)iron, carbonylbis(butadiene)iron, potassium (cyclopentadienyl)dicarbonylferrate, potassium hydridetetracarbonylferrate, sodium hydridetetracarbonylferrate, sodium (cyclopentadienyl)dicarbonylferrate, chloro(cyclopentadienyl)dicarbonyliron, (cyclopentadienyl)methyldicarbonyliron, (cyclopentadienyl)hydridedicarbonyliron, bromo(cyclopentadienyl)carbonyl(trimethylphosphine)iron, (cyclopentadienyl)methylcarbonyl(trimethylphosphine)iron, chloro(cyclopentadienyl){1,2-bis(diphenylphosphino)ethane}iron, tricarbonyl(cyclopentadienyl)iron tetraphenylborate, tricarbonyl(cyclopentadienyl)iron hexafluorophosphate, (cyclopentadienyl)dicarbonyl(tetrahydrofuran)iron tetrafluoroborate, (cyclopentadienyl)dicarbonyl(trimethylphosphine)iron tetrafluoroborate, (cyclopentadienyl)tris(trimethylphosphine)iron bromide, (cyclopentadienyl)dicarbonyl(ethylene)iron hexafluorophosphate, (cyclopentadienyl)dicarbonyl(2-butyne)iron tetrafluoroborate, (cyclopentadienyl)dicarbonyl(dimethylcarbene)iron tetrafluoroborate, (cyclopentadienyl)dicarbonyl(phenylcarbene)iron hexafluoroborate, (cyclopentadienyl)carbonyl(ethoxymethylcarbene)(triphenylphosphine)iron tetrafluoroborate, bis(cyclopentadienyl)iron[ferrocene], iodide trimethyl(ferrocenylmethyl)ammonium, bis(cyclopentadienyl)iron hexafluorophosphate, benzene(cyclopentadienyl)iron hexafluorophosphate, tetracarbonylbis(cyclopentadienyl)diiron, tetracarbonylbis(pentamethylcyclopentadienyl)diiron, enneacarbonyldiiron, chlorobis[(cyclopentadienyl)dicarbonyliron]tetrafluoroborate, bis(pentamethylcyclopentadienyl)bis(disulfide)diiron, (disulfide)hexacarbonyldiiron, sodium octacarbonyldiferrate, dodecacarbonyltriiron, bis{bis(triphenylphosphoranediyl)ammonium}hendecacarbonyltriferrate, bis{bis(triphenylphosphoranediyl)ammonium}tridecacarbonyl tetraferrate, tetrakis(cyclopentadienyl)tetrasulfidetetrairon, (cyclooctatetraene)(cyclooctatetraene)iron, dichlorobis{1,2-bis(diethylphosphino)ethane}iron, chlorohydridebis{bis(1,2-diphenylphosphino)ethane}iron, dihydridebis{1,2-bis(diphenylphosphino)ethane}iron, hydridebis{1,2-bis(diphenylphosphino)ethane}iron, bis{1,2-bis(diphenylphosphino)ethane}ethylene-iron complex, dichlorobis(triphenylphosphine)iron or the like, salts such as iron acetate, iron chloride, iron bromide, iron iodide or the like.

The ruthenium catalysts include supported catalyst such as ruthenium-supported silica, ruthenium-supported alumina, ruthenium-supported carbon or the like, complex catalysts such as pentacarbonylruthenium, dodecacarbonyltriruthenium, tetraethylammonium carbonyldecacarbonyl-μ-hydridetriruthenate, tetra-μ-hydridedodecacarbonyltetraruthenium, bis{bis(triphenylphosphine)}iminium di-μ-carbonyldi-μ₃-carbonyltetradecacarbonylhexaruthenate, tetraethylammonium (μ₆-carbide)tri-μ-carbonyltridecacarbonylhexaruthenate, dihyd ride(dinitrogen)tris(triphenylphosphine)ruthenium, dicarbonyltris(triphenylphosphine)ruthenium, tetracarbonyl(trimethylphosphite)ruthenium, tetracarbonyl(triethylphosphite)ruthenium, tetracarbonyl(triphenylphosphine)ruthenium, pentakis(trimentylphosphite)ruthenium, dichlorotris(triphenylphosphine)ruthenium, diacetatodicarbonylbis(triphenylphosphine)ruthenium, di-μ-chlorobis(chlorotricarbonyl)ruthenium, dichlorotris(triphenylphosphine)ruthenium, carbonylchlorohydridetris(triphenylphosphine)ruthenium, pentakis(trimethylphosphite)ruthenium, tris(acetylacetonato)ruthenium, diacetatodicarbonylbis(triphenylphosphine)ruthenium, dinitratocarbonylbis(triphenylphosphine)ruthenium, dihydridetetrakis(triphenylphosphine)ruthenium, tetrahydridetris(triphenylphosphine)ruthenium, dihydride(trifluorophosphine)tris(triphenylphosphine)ruthenium, acetatohydridetris(triphenylphosphine)ruthenium, hydridenitrosyltris(triphenylphosphine)ruthenium, trichloronitrosylbis(triphenylphosphine)ruthenium, tetrafluoroboric acid chlorodinitrosylbis(triphenylphosphine)ruthenium, dichlorobis(acetonitrile)bis(triphenylphosphine)ruthenium, dichlorotetrakis(isocyanated t-butyl)ruthenium, bis(cyclopentadienyl)ruthenium, bis(pentamethylcyclopentadienyl)ruthenium, (cyclopentadienyl)(pentamethylcyclopentadienyl)ruthenium, tetracarbonylbis(cyclopentadienyl)diruthenium, tetracarbonylbis(pentamethylcyclopentadienyl)diruthenium, dichloro(pentamethylcyclopentadienyl)ruthenium, chloro(cyclopentadienyl)bis(triphenylphosphine)ruthenium, hydride(cyclopentadienyl)bis(triphenylphosphine)ruthenium, bromo(cyclopentadienyl)bis(triphenylphosphine)ruthenium, chloro(cyclopentadienyl)bis(triphenylphosphine)ruthenium, bromo(cyclopentadienyl)bis(trimethylphosphine)ruthenium, cholro(cyclopentadienyl)bis(trimethylphosphine)ruthenium, cyclopentadienyltris (trimethylphosphine)ruthenium hexafluorophosphate, chlorodicarbonyl(cyclopentadienyl)ruthenium, hydride(cyclopentadienyl)(1,5-cyclooctadiene)ruthenium, chloro(cyclopentadienyl)(1,5-cyclooctadiene)ruthenium, bromo(cyclopentadienyl)(1,5-cyclooctadiene)ruthenium, chlorocarbonyl(pentamethylcyclopentadienyl)ruthenium, bromodicarbonyl(pentamethylcyclopentadienyl)ruthenium, iododicarbonyl(pentamethylcyclopentadienyl)ruthenium, tricarbonyl(pentamethylcyclopentadienyl)ruthenium tetrafluoroborate, dicarbonyl(hydroxymethyl)(pentamethylcyclopentadienyl)ruthenium, chloro(pentamethylcyclopentadienyl)(1,5-cyclooctadiene)ruthenium, chloro(pentamethylcyclopentadienyl)(norbornadiene)ruthenium, chloro(pentamethylcyclopentadienyl)bis(methyidiphenylphosphine)ruthenium, chloro(pentamethylcyclopentadienyl)bis(dimethylphenylphosphine)ruthenium, chloro(pentamethylcyclopentadienyl)bis(triethylphosphine)ruthenium, chloro(pentamethylcyclopentadienyl)(1,2-diphenylphosphinoethane)ruthenium, chloro(pentamethylcyclopentadienyl)(1,3-diphenylphosphinopropane)ruthenium, chloro(pentamethylcyclopentadienyl)(1,4-diphenylphosphinobutane)ruthenium, chloro(pentamethylcyclopentadienyl)(1,5-diphenylphosphinopentane)ruthenium, chloro(pentamethylcyclopentadienyl)(1,6-diphenylphosphinohexane)ruthenium, dicarbonylcyclopentadienylruthenium dimer, dichloro(pentamethylcyclopentadienyl)(triphenylphosphine)ruthenium, trichloro(pentamethylcyclopentadienyl)ruthenium, dihydridetetrakis(triphenylphosphine)ruthenium, trihydride(pentamethylcyclopentadienyl)(triphenylphosphine)ruthenium, dichloro(allyl) (cyclopentadienyl)ruthenium, dichloro(allyl) (pentamethylcyclopentadienyl)ruthenium, tricarbonyl(cyclooctatetraene)ruthenium, chlorohydridetris(triphenylphosphine)ruthenium, tricarbonylbis(triphenylphosphine)ruthenium, tricarbonyl(1,5-cyclooctadiene)ruthenium, (cyclooctatriene)(cyclooctadiene)ruthenium, bis(allyl)(norbornadiene)ruthenium or the like, or ruthenium chloride, ruthenium oxide, ruthenium black or the like.

The cobalt catalysts include complex catalysts such as Raney cobalt, or octacarbonyidicobalt, dodecacarbonyltetracobalt, hydridetetracarbonylcobalt, cyclopentadienyidicarbonylcobalt, chlorotris(triphenylphosphine)cobalt, cobaltcene or the like, or salts such as cobalt acetate, cobalt chloride, cobalt bromide, cobalt iodide, cobalt nitrate or the like.

The nickel catalysts include solid and supported catalysts such as Raney nickel catalyst, nickel-supported silica, nickel-supported alumina, nickel-supported carbon or the like, complex catalysts such as tetracarbonylnickel, dichlorobis(triphenylphosphine)nickel, tetrakis(triphenylphosphine)nickel, tetrakis(triphenylphosphite)nickel, bis(1,5-cyclooctadiene)nickel, nickelocene, bis(pentamethylcyclopentadienyl)nickel, bis(triphenylphosphine)nickeldicarbonyl or the like, nickel acetate, nickel chloride, nickel bromide, nickel oxide or the like.

The palladium catalysts include solid and supported catalysts such as Raney palladium, palladium-supported silica catalyst, palladium-supported alumina catalyst, palladium-supported carbon catalyst, palladium-supported barium sulfate catalyst, palladium-supported zeolite catalyst, palladium-supported silica/alumina catalyst or the like, complex catalysts such as dichlorobis(triphenylphosphine) palladium, dichlorobis(trimethylphosphine)palladium, dichlorobis(tributylphosphine)palladium, bis(tricyclohexylphosphine)palladium, tetrakis(triethylphosphite)palladium, bis(cycloocta-1,5-diene)palladium, tetrakis(triphenylphosphine)palladium, dicarbonylbis(triphenylphosphine)palladium, carbonyltris(triphenylphosphine)palladium, dichlorobis(benzonitryl)palladium, dichloro(1,5-cyclooctadiene)palladium or the like, or palladium chloride, palladium acetate, palladium oxide or the like.

The rhodium catalysts include supported catalysts such as rhodium-supported silica catalyst, rhodium-supported alumina catalyst, rhodium-supported carbon catalyst or the like, complex catalysts such as chlorotris(triphenylphosphine) rhodium, hexadecacarbonylhexarhodium, dodecacarbonyltetrarhodium, dichlorotetracarbonyldirhodium, hydridetetracarbonylrhodium, hydridecarbonyltris(triphenylphosphine)rhodium, hydride(triphenylphosphine)rhodium, dichlorobis(cyclooctadiene)dirhodium, dicarbonyl(pentamethylcyclopentadienyl)rhodium, cyclopentadienylbis(triphenylphosphine)rhodium, dichlorotetrakis(allyl)dirhodium or the like, or rhodium chloride, rhodium oxide or the like.

The platinum catalysts include supported catalysts such as platinum-supported silica catalyst, platinum-supported alumina catalyst, platinum-supported carbon catalyst or the like, complex catalysts such as dichlorobis(triphenylphosphine) platinum, dichlorobis(trimethylphosphine)platinum, dichlorobis(tributylphosphine)platinum, tetrakis(triphenylphosphine)platinum, tetrakis(triphenylphosphite)platinum, tris(triphenylphsophine)platinum, dicarbonylbis(triphenylphosphine)platinum, carbonyltris(triphenylphosphine)platinum, cis-bis(benzonitryl)dichloroplatinum, bis(1,5-cyclooctadiene)platinum or the like, or platinum chloride, platinum oxide (Addams catalyst), platinum black or the like.

Among them, catalysts in which metal is iron, ruthenium, palladium or cobalt, rhodium are preferable, and catalysts in which metal is iron, ruthenium, palladium or platinum are particularly preferable. These catalysts may be used singly or in a combination thereof.

The used amount of the catalyst comprising a Group VIII metal of the Periodic Table is preferably 0.001 to 50 mol %, more preferably 0.01 to 30 mol % based on 2-nitrobenzylcarbonyl compound being a substrate.

In the reaction, some additives can be optionally coexistent. The additives include for example monodentate or multidentate tertiary phosphines such as trimethylphosphine, triethylphosphine, tributylphosphine, tricyclohexylphosphine, triphenylphosphine, tris(paratolyl)phosphine, tris(2,6-dimethylphenyl)phosphine, sodium diphenylphosphinobenzene-3-sulfonate, bis(3-sulfonatephenyl)phosphinobenzene sodium salt, 1,2-bis(diphenylphosphino)ethane, 1,3-bis(diphenylphosphino)propane, 1,4-bis(diphenylphosphino)butane, 1,1′-bis(diphenylphosphino)ferrocene, tris(3-sulfonatephenyl)phosphine sodium salt or the like, phosphites such as triethylphosphite, tributylphosphite, triphenylphosphite, tris(2,6-dimethylphenyl)phosphite or the like, phosphonium salts such as triphenylmethylphosphonium iodide, triphenylmethylphosphonium bromide, triphenylmethylphosphonium chloride, triphenylallylphosphonium iodide, triphenylallylphosphonium bromide, triphenylallylsulfonium chloride, tetraphenylphosphonium iodide, tetraphenylphosphonium bromide, tetraphenylphosphonium chloride or the like, phostates such as triphenyl phosphate, trimethyl phosphate, triethyl phosphate, triallyl phosphate or the like, nitriles such as benzonitrile, acetonitrile or the like, ketones such as acetylacetone or the like, unsaturated hydrocarbons such as cyclopentadiene, pentamethylcyclopentadiene, 1,5-cyclooctadiene, norbornadiene or the like, nitrogen containing hetrocyclic compounds such as pyridine, 2-picoline, 3-picoline, 4-picoline, 2,2-bipyridyl, terpyridine, 1,10-phenathrorine, 8-hydroxyquinoline, bisoxazolynylpyridine (Pybox), 1,4-dimethylpyrazole, 1,3,5-trimethylpyrazole, pyrmidine, pyrazine or the like, inorganic compounds such as tin (II) chloride, copper (I) chloride, copper (I) bromide, sodium chloride, sodium bromide, sodium iodide, potassium chloride, potassium bromide, sodium hydroxide, potassium hydroxide or the like.

The added amount of the additives are variously varied depending on the purpose or use, and are preferably 0.001 to 500 mol % and more preferably 0.01 to 200 mol % based on 2-nitrobenzylcarbonyl compound being a substrate.

The used amount of carbon monoxide is sufficient if it is finally supplied in a stoichiometric amount to be used in the reaction. It is preferable to conduct the reaction in a total pressure in the reaction system of 0.5 to 300 kgf/cm² and a carbon monoxide partial pressure of 0.2 to 100 kgf/cm². It is able to compensate the differential pressure between the total pressure and the carbon monoxide partial pressure with a gas such as nitrogen, argon, helium, carbon dioxide or the like that is inert to the pressure of the solvent itself or the reaction.

In order to smoothly proceed the reaction including dispersing and mixing each agents, it is preferable to carry out the reaction in a state diluted with a solvent. The solvent used for the reaction is not specifically limited so long as it is an inert solvent for the present reaction, and for example includes ethers such as diethyl ether, methyl-t-butyl ether, tetrahydrofuran, diethylether, dimethoxymethane, diethoxymethane, ethylene glycol dimethyl ether, ethylene glycol diethyl ether, ethylene glycol dibutyl ether, diethylene glycol dimethyl ether, diethylene glycol diethyl ether, diethylene glycol dibutyl ether, triethylene glycol dimethyl ether, 1,4-dioxane and the like, alcohols such as methanol, ethanol, 1-propanol, 2-propanol, 1-butanol, 2-butanol, isobutanol, 2-methyl-2-propanol, methylcellosolve, ethylcellosolve, i-propylcellosolve, diethylene glycol monomethyl ether, diethylene glycol monoethyl ether, diethylene glycol mono butyl ether, cyclohexanol, benzylalcohol and the like, ketones such as acetone, methyl ethyl ketone, diethyl ketone, 2-pentanone, methyl isobutyl ketone, cyclohexanone and the like, aliphatic hydrocarbons such as pentane, hexane, cyclohexane, methylcyclohexane, heptane, octane, decane and the like, halogenated hydrocarbons such as chloroform, carbon tetrachloride, dichloroethane, tetrachloroethylene and the like, aromatic hydrocarbones such as benzene, toluene, xylene, chlorobenzene, o-dichlorobenzene, m-dichlorobenzene, p-dichlorobenzene, nitrobenzene, tetrahydronaphthalene and the like, nitriles such as acetonitrile, propionitrile and the like, esters such as methyl acetate, ethyl acetate, butyl acetate, ethyl propionate and the like, amides such as N,N-dimethylformamide, N,N-dimethylacetamide, N-methylpyrrolidone and the like, ureas such as 1,3-dimethylimidazolidinone, N,N,N′,N′-tetramethylurea and the like, pyridines such as pyridine, 2-picoline, 3-picoline, 4-picoline, 5-ethyl-2-picoline and the like, or water. These solvents can be used singly or in a combination thereof.

The present reaction can be conducted in a wide temperature range. However, it is preferable to conduct the reaction generally in a temperature of 50 to 400° C., particularly in a temperature of 80 to 300° C. from the viewpoint of economical production including the used amount of the reaction agents.

The reaction time is varied depending on the amount or concentration of the agents used, reaction temperature and the like. However, it is preferable to set the condition of the reaction so as to conclude the reaction in a range of 0.1 to 30 hours, preferably 0.5 to 20 hours.

It is preferable to carry out the present reaction by use of a pressurized reactor such as autoclave or the like. The reaction can be conducted in a batch type or a continuous type, and the type can be selected depending on the substrate concentration, conversion rate, producibility and the like that are required for the reaction.

After the conclusion of the reaction, the solvent is distilled off if required, and then an aimed product is directly obtained by distillation, or water and solvents immiscible in water are added in a crude reaction product, fully washed, and then the organic phase is subjected to conventional treatment such as distillation, recrystallization, column chromatography or the like to purify and isolate an aimed indole derivative.

Hereinafter, the present invention is further described according to examples to which the present invention is not limited.

### REFERENCE EXAMPLE 1

### Production of 2-[4-fluoro-2-nitrophenyl]-3-hydroxy-2-butenoic acid methyl ester (2-[4-fluoro-2-nitrophenyl]-3-oxobutanoic acid methyl ester)

A mixed suspension of 19.0 g of powdery potassium carbonate, 10.0 g of 2,5-difluoronitrobenzene and 50 mL of N,N-dimethylformamide was warmed to 50° C., and then 8.39 g of methyl acetoacetate was added thereto. Thereafter, the mixture was stirred under nitrogen atmosphere at 49 to 51° C. for 19 hours, and then it was stood to cool to 22° C. 150 mL of toluene was added to the reaction mixture, and the resulting mixture was added to 300 mL of cold water of 10° C. The toluene phase was removed, and then the extractive process was conducted twice by adding 150 mL of toluene to the aqueous phase. The resulting toluene phases were mixed, washed with 150 mL of water three times, and then the extractive process to the aqueous phase was conducted by adding 150 mL of 5% sodium hydroxide aqueous solution (twice). 35 mL of 35% hydrochloric acid was added to the resulting aqueous phase to adjust to pH 3, and the extractive process with 150 mL of toluene was conducted twice. The resulting toluene solution was washed with 200 mL of water three times, and then filtered through a liquid phase separation filter paper, and washed by pouring 20 mL of toluene on the filter paper and the solvent was distilled off and dried to obtain 12.1 g (yield 76%) of 2-[4-fluoro-2-nitrophenyl]-3-hydroxy-2-butenoic acid methyl ester (2-[4-fluoro-2-nitrophenyl]-3-oxobutanoic acid methyl ester) (keto form:enol form in CDCl₃=1:9).

### REFERENCE EXAMPLE 2

### Production of 1-(4-fluoro-2-nitorphenyl)acetone from 2-[4-fluoro-2-nitrophenyl]-3-hydroxy-2-butenoic acid methyl ester (2-[4-fluoro-2-nitrophenyl]-3-oxobutanoic acid methyl ester)

At room temperature, 110 g of acetic acid was added to 18.1 g of 2-[4-fluoro-2-nitrophenyl]-3-hydroxy-2-butenoic acid methyl ester (2-[4-fluoro-2-nitrophenyl]-3-oxobutanoic acid methyl ester), and then 26.0 g of 50% sulfuric acid aqueous solution was added. The mixture was gradually heated with stirring, and reacted finally at a reflux temperature for 4.7 hours. Water and acetic acid were partially distilled off from the reaction mixture, and 100 mL of toluene was added thereto, and the resulting mixture was gradually put in 100 mL of water. After separating the toluene phase, 100 mL of toluene was further added to the aqueous phase and the toluene phase was separated again. The resulting toluene phases were washed 100 mL of water four times, and filtered through celite. The solvent was distilled off under a reduced pressure, and 100 mL of n-hexane was added to obtain a slurry. The crystal obtained by filtrating the slurry was dried under a reduced pressure to obtain 12.8 g (yield 92%) of 1-[4-fluoro-2-nitrophenyl]acetone.

### EXAMPLE 1

### Synthesis of 6-fluoro-2-methylindole

In a stainless steel autoclave having an internal volume of 100 mL, 1.0 g (5.1 mmol) of 4-fluoro-2-nitrophenylacetone, 72 mg (4 mol %) of cyclopentadienyidicarbonyliron (dimer) and 40 g of toluene were placed under nitrogen atmosphere. After the atmosphere inside of the reactor was substituted by nitrogen gas (10 kgf/cm²) three times, carbon monoxide gas was injected (50 kgf/cm²), and the resulting mixture was reacted at a temperature of 120° C. for 5 hours. After cooling, the reaction solution was subjected to quantitative analysis with liquid chromatography. As a result of it, it was confirmed that 4-fluoro-2-nitrophenylacetone was completely disappeared and 0.64 g (yield 95.0%) of 6-fluoro-2-methylindole was formed.

### EXAMPLE 2

### Synthesis of 6-fluoro-2-methylindole

In a stainless steel autoclave having an internal volume of 100 mL, 1.0 g (5.1 mmol) of 4-fluoro-2-nitrophenylacetone, 178 mg (5 mol %) of dichlorobis(triphenylphosphine)palladium, 0.48 g tin (II) chloride and 40 g of 1,4-dioxane were placed under nitrogen atmosphere. After the atmosphere inside of the reactor was substituted by nitrogen gas (10 kgf/cm²) three times, carbon monoxide gas was injected (20 kgf/cm²), and the resulting mixture was reacted at a temperature of 120° C. for 9 hours. After cooling, the reaction solution was subjected to quantitative analysis with liquid chromatography. As a result of it, it was confirmed that 4-fluoro-2-nitrophenylacetone was consumed by 98% and 0.61 g (yield 91.0%) of 6-fluoro-2-methylindole was formed.

### EXAMPLE 3

### Synthesis of 6-fluoro-2-methylindole

The reaction and treatment were conducted in an entirely similar manner as those in Example 2 except that dichlorobis(triphenylphosphine)palladium as a catalyst was replaced by 295 mg (5 mol %) of tetrakis(triphenylphosphine)palladium. It was confirmed that the conversion rate of 4-fluoro-2-nitrophenylacetone was 100% and 0.60 g (yield 89.0%) of 6-fluoro-2-methylindole was formed.

### EXAMPLE 4

### Synthesis of 6-fluoro-2-methylindole

In a stainless steel autoclave having an internal volume of 100 mL, 1.0 g (5.1 mmol) of 4-fluoro-2-nitrophenylacetone, 130 mg (4 mol %) of trirutheniumdodecacarbonyl and 40 g of toluene were placed under nitrogen atmosphere. After the atmosphere inside of the reactor was substituted by nitrogen gas (10 kgf/cm²) three times, carbon monoxide gas was injected (50 kgf/cm²), and the resulting mixture was reacted at a temperature of 180° C. for 4 hours. After cooling, the reaction solution was subjected to quantitative analysis with liquid chromatography. As a result of it, it was confirmed that the conversion rate of 4-fluoro-2-nitrophenylacetone was 91 % and 0.53 g (yield 79.0%) of 6-fluoro-2-methylindole was formed.

### EXAMPLE 5

### Synthesis of 6-fluoro-2-methylindole

In a stainless steel autoclave having an internal volume of 100 mL, 1.0 g (5.1 mmol) of 4-fluoro-2-nitrophenylacetone, 130 mg (4 mol %) of trirutheniumdodecacarbonyl, 397 mg of 2,2′-bipyridyl and 40 g of toluene were placed under nitrogen atmosphere, and the reaction was conducted similarly to Example 4. After the reaction, the reaction solution was subjected to quantitative analysis with liquid chromatography. As a result of it, it was confirmed that the conversion rate of 4-fluoro-2-nitrophenylacetone was 100% and 0.65 g (yield 97.0%) of 6-fluoro-2-methylindole was formed.

### EXAMPLE 6

### Synthesis of 6-fluoro-2-methylindole

In a stainless steel autoclave having an internal volume of 100 mL, 1.0 g (5.1 mmol) of 4-fluoro-2-nitrophenylacetone, 130 mg (4 mol %) of trirutheniumdodecacarbonyl, 7 mg of 1,10-phenanthroline and 40 g of toluene were placed under nitrogen atmosphere, and the reaction was conducted similarly to Example 4. After the reaction, the reaction solution was subjected to quantitative analysis with liquid chromatography. As a result of it, it was confirmed that the conversion rate of 4-fluoro-2-nitrophenylacetone was 100% and 0.63 g (yield 94.0%) of 6-fluoro-2-methylindole was formed.

### EXAMPLE 7

### Synthesis of 6-fluoro-2-methylindole

In a stainless steel autoclave having an internal volume of 100 mL, 1.0 g (5.1 mmol) of 4-fluoro-2-nitrophenylacetone, 102.5 mg (4 mol %) of dichlorobis(triphenylphosphine)platinum, 0.48 g of tin(II) chloride and 30 g of 1,4-dioxane were placed under nitrogen atmosphere. After the atmosphere inside of the reactor was substituted by nitrogen gas (10 kgf/cm²) three times, carbon monoxide gas was injected (50 kgf/cm²), and the resulting mixture was reacted at a temperature of 120° C. for 8 hours. After cooling, the reaction solution was subjected to quantitative analysis with liquid chromatography. As a result of it, it was confirmed that the conversion rate of 4-fluoro-2-nitrophenylacetone was 92% and 0.29 g (yield 44.0%) of 6-fluoro-2-methylindole was formed.

### EXAMPLE 8

### Synthesis of 6-fluoro-2-methylindole

In a stainless steel autoclave having an internal volume of 100 mL, 1.0 g (5.1 mmol) of 4-fluoro-2-nitrophenylacetone, 0.05 g of 5% palladium supported on active carbon [manufactured by N.E. CHEMCAT CORPORATION (50% water-containing product)], 0.48 g of tin (II) chloride, 0.20 g of triphenylphosphine and 40 g of 1,4-dioxane were placed under nitrogen atmosphere. After the atmosphere inside of the reactor was substituted by nitrogen gas (10 kgf/cm²) three times, carbon monoxide gas was injected (20 kgf/cm²), and the resulting mixture was reacted at a temperature of 120° C. for 4 hours. After cooling, the reaction solution was subjected to quantitative analysis with liquid chromatography. As a result of it, it was confirmed that the conversion rate of 4-fluoro-2-nitrophenylacetone was 33% and 0.07 g (yield 10.0%) of 6-fluoro-2-methylindole was formed.

### EXAMPLE 9

### Synthesis of 2-methylindole

In a stainless steel autoclave having an internal volume of 100 mL, 0.91 g (5.1 mmol) of 2-nitrophenylacetone, 72 mg (4 mol %) of cyclopentadienyidicarbonyliron (dimer) and 40 g of toluene were placed under nitrogen atmosphere. After the atmosphere inside of the reactor was substituted by nitrogen gas (10 kgf/cm²) three times, carbon monoxide gas was injected (30 kgf/cm²), and the resulting mixture was reacted at a temperature of 120° C. for 5 hours. After cooling, the reaction solution was subjected to quantitative analysis with liquid chromatography. As a result of it, it was confirmed that 2-nitrophenylacetone remained by 3% and 0.60 g (yield 89.0%) of 2-methylindole was formed.

### EXAMPLE 10

### Synthesis of 5-chloro-2-methylindole

In a stainless steel autoclave having an internal volume of 100 mL, 1.09 g (5.1 mmol) of 5-chloro-2-nitrophenylacetone, 72 mg (4 mol %) of cyclopentadienyidicarbonyliron (dimer) and 40 g of toluene were placed under nitrogen atmosphere. After the atmosphere inside of the reactor was substituted by nitrogen gas (10 kgf/cm²) three times, carbon monoxide gas was injected (30 kgf/cm²), and the resulting mixture was reacted at a temperature of 120° C. for 5 hours. After cooling, the reaction solution was subjected to quantitative analysis with liquid chromatography. As a result of it, it was confirmed that 5-chloro-2-nitrophenylacetone was completely disappeared. Thereafter, post-treatment was conducted, and the main product was isolated with silica gel chromatography to obtain 0.79 g (yield 94.0%) of 6-fluoro-2-methylindole.

### EXAMPLE 11

### Synthesis of 6-bromo-2-methylindole

In a stainless steel autoclave having an internal volume of 100 mL, 1.31 g (5.1 mmol) of 4-bromo-2-nitrophenylacetone, 72 mg (4 mol %) of cyclopentadienyidicarbonyliron (dimer) and 40 g of toluene were placed under nitrogen atmosphere. After the atmosphere inside of the reactor was substituted by nitrogen gas (10 kgf/cm²) three times, carbon monoxide gas was injected (30 kgf/cm²), and the resulting mixture was reacted at a temperature of 120° C. for 5 hours. After cooling, the reaction solution was subjected to quantitative analysis with liquid chromatography. As a result of it, it was confirmed that 5-fluoro-2-nitrophenylacetone was completely disappeared. The isolation treatment as mentioned above provided 1.00 g (yield 93.0%) of 6-bromo-2-methylindole.

### EXAMPLE 12

### Synthesis of 2-methyl-6-trifluoromethylindole

In a stainless steel autoclave having an internal volume of 100 mL, 1.26 g (5.1 mmol) of 2-nitro4-trifluoromethylphenylacetone, 72 mg (4 mol %) of cyclopentadienyldicarbonyliron (dimer) and 40 g of toluene were placed under nitrogen atmosphere. After the atmosphere inside of the reactor was substituted by nitrogen gas (10 kgf/cm²) three times, carbon monoxide gas was injected (30 kgf/cm²), and the resulting mixture was reacted at a temperature of 120° C. for 5 hours. After cooling, the reaction solution was subjected to quantitative analysis with liquid chromatography. As a result of it, it was confirmed that 5-fluoro-2-nitrophenylacetone was completely disappeared and 0.87 g (yield 86.0%) of 2-methyl-6-trifluoromethylindole was formed.

### EXAMPLE 13

### Synthesis of 2-methyl-6-nitroindole

In a stainless steel autoclave having an internal volume of 100 mL, 1.14 g (5.1 mmol) of 2,5-dinitrophenylacetone, 72 mg (4 mol %) of cyclopentadienyldicarbonyliron (dimer) and 40 g of toluene were placed under nitrogen atmosphere. After the atmosphere inside of the reactor was substituted by nitrogen gas (10 kgf/cm²) three times, carbon monoxide gas was injected (50 kgf/cm²), and the resulting mixture was reacted at a temperature of 150° C. for 3 hours. After cooling, the reaction solution was subjected to quantitative analysis with liquid chromatography. As a result of it, the conversion rate of 2,5-dinitrophenylacetone was 52%, 2-methyl-6-nitroindole as a main product was obtained in a yield of 22% and 6-amino-2-methylindole was not formed at all.

### EXAMPLE 14

### Synthesis of 6-fluoro-3-methoxycarbonyl-2-methylindole

In a stainless steel autoclave having an internal volume of 100 mL, 1.30 g (5.1 mmol) of 2-[4-fluoro-2-nitrophenyl]-3-hydroxy-2-butenoic acid methyl ester, 72 mg (4 mol %) of cyclopentadienyldicarbonyliron (dimer) and 20 g of toluene were placed under nitrogen atmosphere. After the atmosphere inside of the reactor was substituted by nitrogen gas (10 kgf/cm²) three times, carbon monoxide gas was injected (50 kgf/cm²), and the resulting mixture was reacted at a temperature of 150° C. for 5 hours. After cooling, the reaction solution was subjected to quantitative analysis with liquid chromatography. As a result of it, the conversion rate of 2-[4-fluoro-2-nitrophenyl]-3-hydroxy-2-butenoic acid methyl ester being a starting material was 84.2% and de-methoxycarbonylated or de-acetylated products of the starting material were formed by about 45%. It was confirmed that as indole derivatives, 0.26 g (yield 25.0%) of 6-fluoro-3-methoxycarbonyl-2-methylindole and 0.15 g (yield 14.0%) of 3-acetyl-6-fluoro-2-methoxyindole were formed.

### EXAMPLE 15

### Synthesis of 6-fluoro-3-methoxycarbonyl-2-methylindole

In a stainless steel autoclave having an internal volume of 100 mL, 1.30 g (5.1 mmol) of 2-[4-fluoro-2-nitrophenyl]-3-hydroxy-2-butenoic acid methyl ester, 130 mg (4 mol %) of trirutheniumdodecacarbonyl catalyst and 20 g of toluene were placed under nitrogen atmosphere. After the atmosphere inside of the reactor was substituted by nitrogen gas (10 kgf/cm²) three times, carbon monoxide gas was injected (50 kgf/cm²), and the resulting mixture was reacted at a temperature of 180° C. for 5 hours. After cooling, the reaction solution was subjected to quantitative analysis with liquid chromatography. As a result of it, it was confirmed that 2-[4-fluoro-2-nitrophenyl]-3-hydroxy-2-butenoic acid methyl ester being a starting material was completely disappeared and 0.75 g (yield 71.0%) of 6-fluoro-3-methoxycarbonyl-2-methylindole and 0.11 g (yield 16%) of 6-fluoro-2-methoxyindole were formed.

### EXAMPLE 16

### Synthesis of 3-acetyl-6-fluoro-2-methylindole

In a stainless steel autoclave having an internal volume of 100 mL, 1.22 g (5.1 mmol) of 3-(4-fluoro-2-nitrophenyl)4-hydroxy-3-penten-2-one, 72 mg (4 mol %) of cyclopentadienyidicarbonyliron (dimer) and 40 g of toluene were placed under nitrogen atmosphere. After the atmosphere inside of the reactor was substituted by nitrogen gas (10 kgf/cm²) three times, carbon monoxide gas was injected (60 kgf/cm²), and the resulting mixture was reacted at a temperature of 120° C. for 5 hours, and further at an increased temperature of 170° C. for 2 hours. After cooling, the reaction solution was subjected to quantitative analysis with liquid chromatography. As a result of it, it was confirmed that 3-(4-fluoro-2-nitrophenyl)-4-hydroxy-3-penten-2-one being a starting material was completely disappeared and 0.11 g (yield 11%) of 6-fluoro-3-methoxycarbonyl-2-methylindole and 0.07 g (yield 10%) of 6-fluoro-2-methylindole were formed.

### EXAMPLE 17

### Synthesis of 2,3-dimethylindole

In a stainless steel autoclave having an internal volume of 100 mL, 0.99 g (5.1 mmol) of 1-methyl-1-(o-nitrophenyl)acetone, 72 mg (4 mol %) of cyclopentadienyldicarbonyliron (dimer) and 20 g of toluene were placed under nitrogen atmosphere. After the atmosphere inside of the reactor was substituted by nitrogen gas (10 kgf/cm²) three times, carbon monoxide gas was injected (30 kgf/cm²), and the resulting mixture was reacted at a temperature of 120° C. for 8 hours. After cooling, the reaction solution was subjected to quantitative analysis with liquid chromatography. As a result of it, the conversion rate of 1-methyl-1-(o-nitrophenyl)acetone was 100% and 0.67 g (yield 91%) of 2,3-dimethylindole was formed.

### EXAMPLE 18

### Synthesis of 5-benzyloxy-6-fluoro-2-methylindole

In a stainless steel autoclave having an internal volume of 100 mL, 1.55 g (5.1 mmol) of 5-benzyloxy4-fluoro-2-nitrophenylacetone, 72 mg (4 mol %) of cyclopentadienyldicarbonyliron (dimer) and 26 g of toluene were placed under nitrogen atmosphere. After the atmosphere inside of the reactor was substituted by nitrogen gas (10 kgf/cm²) three times, carbon monoxide gas was injected (30 kgf/cm²), and the resulting mixture was reacted at a temperature of 120° C. for 5 hours and 30 minutes. After cooling, the reaction solution was subjected to quantitative analysis with liquid chromatography. As a result of it, the conversion rate of 5-benzyloxy-4-fluoro-2-nitrophenylacetone was 100% and 1.07 g (yield 82%) of 5-benzyloxy-6-fluoro-2-methylindole as a main product was formed.

### COMPARATIVE EXAMPLE 1

### Synthesis of 6-fluoro-2-methylindole

In a reaction flask the atmosphere inside of which was substituted by nitrogen, 1.00 g (5.1 mmol) of 4-fluoro-2-nitrophenylacetone, 10 g of 2-ethoxyetanol and 0.05 g of 5% palladium supported on active carbon [manufactured by N.E. CHEMCAT CORPORATION (50% water-containing product)] were placed, hydrogen gas was supplied therein at ordinary pressure and 20° C., and the resulting mixture was reacted for 24 hours. After confirming the disappearance of 4-fluoro-2-nitrophenylacetone with liquid chromatography, the atmosphere was substituted by nitrogen, and the catalyst was filtered off through celite. The reaction solution was subjected to quantitative analysis with liquid chromatography, and as a result of it, it was confirmed that 0.13 g (yield 17%) of 6-fluoro-2-methylindole was formed, and further 6-fluoro-1-hydroxy-2-methylindole and 6-fluoro-2-methylindoline were formed in a yield of 55% and 11%, respectively.

### COMPARATIVE EXAMPLE 2

### Synthesis of 6-fluoro-2-methylindole

In a reaction flask the atmosphere inside of which was substituted by nitrogen, 1.00 g (5.1 mmol) of 4-fluoro-2-nitrophenylacetone, 10 g of 1-butanol and 0.05 g of 5% palladium supported on active carbon [manufactured by N.E. CHEMCAT CORPORATION (50% water-containing product)] were placed, hydrogen gas was supplied therein at ordinary pressure and 100° C., and the resulting mixture was reacted for 24 hours. After confirming the disappearance of 4-fluoro-2-nitrophenylacetone with liquid chromatography, the atmosphere was substituted by nitrogen, and the catalyst was filtered off through celite. The reaction solution was subjected to quantitative analysis with liquid chromatography, and as a result of it, it was confirmed that 0.53 g (yield 70%) of 6-fluoro-2-methylindole was formed, and further 6-fluoro-1-hydroxy-2-methylindole and 6-fluoro-2-methylindoline were formed in a yield of 3% and 25%, respectively.

### COMPARATIVE EXAMPLE 3

### Synthesis of 5-chloro-2-methylindole

In a reaction flask the atmosphere inside of which was substituted by nitrogen, 1.09 g (5.1 mmol) of 5-chloro-2-nitrophenylacetone, 10 g of 1-butanol and 0.054 g of 5% palladium supported on active carbon [manufactured by N.E. CHEMCAT CORPORATION (50% water-containing product)] were placed, hydrogen gas was supplied therein at ordinary pressure and 90° C., and the resulting mixture was reacted for 5 hours. After confirming the disappearance of 4-bromo-2-nitrophenylacetone with liquid chromatography, the atmosphere was substituted by nitrogen, and the catalyst was filtered off through celite. The reaction solution was subjected to quantitative analysis with liquid chromatography, and as a result of it, 5-chloro-2-methylindole was not obtained at all, and a mixture composed of many products including 2-methylindole that bromine atom was eliminated, and the like was obtained.

### COMPARATIVE EXAMPLE 4

### Synthesis of 6-bromo-2-methylindole

In a reaction flask the atmosphere inside of which was substituted by nitrogen, 1.31 g (5.1 mmol) of 4-bromo-2-nitrophenylacetone, 10 g of 1-butanol and 0.065 g of 5% palladium supported on active carbon [manufactured by N.E. CHEMCAT CORPORATION (50% water-containing product)] were placed, hydrogen gas was supplied therein at ordinary pressure and 90° C., and the resulting mixture was reacted for 5 hours. After confirming the disappearance of 4-bromo-2-nitrophenylacetone with liquid chromatography, the atmosphere was substituted by nitrogen, and the catalyst was filtered off through celite. The reaction solution was subjected to quantitative analysis with liquid chromatography, and as a result of it, 6-bromo-2-methylindole was not obtained at all, and a mixture composed of many products including 2-methylindole that bromine atom was eliminated, and the like was obtained.

### COMPARATIVE EXAMPLE 5

### Synthesis of 2-methyl-6-nitroindole

In a reaction flask the atmosphere inside of which was substituted by nitrogen, 1.14 g (5.1 mmol) of 2,5-dinitrophenylacetone, 10 g of 1-butanol and 0.057 g of 5% palladium supported on active carbon [manufactured by N.E. CHEMCAT CORPORATION (50% water-containing product)] were placed, hydrogen gas was supplied therein at ordinary pressure and 90° C., and the resulting mixture was reacted for 5 hours. After confirming the disappearance of 2,5-dinitrophenylacetone with liquid chromatography, the atmosphere was substituted by nitrogen, and the catalyst was filtered off through celite. The reaction solution was subjected to quantitative analysis with liquid chromatography, and as a result of it, 2-methyl-6-nitroindole was not obtained at all, and 6-amino-2-methylindole that nitro group was reduced was obtained as a main product in a yield of 75%.

### COMPARATIVE EXAMPLE 6

### Synthesis of 5-benzyloxy-6-fluoro-2-methylindole

In a reaction flask the atmosphere inside of which was substituted by nitrogen, 1.55 g (5.1 mmol) of 5-benzyloxy4-fluoro-2-nitrophenylacetone, 10 g of 1-butanol and 0.078 g of 5% palladium supported on active carbon [manufactured by N.E. CHEMCAT CORPORATION (50% water-containing product)] were placed, hydrogen gas was supplied therein at ordinary pressure and 90° C., and the reaction was ceased after confirming the disappearance of the starting material with liquid chromatography. After cooling, the atmosphere was substituted by nitrogen, and the catalyst was filtered off through celite. The reaction solution was subjected to quantitative analysis with liquid chromatography, and as a result of it, 5-benzyloxy-6-fluoro-2-methylindole was not obtained at all, and 6-fluoro-5-hydroxy-2-methylindole that benzyl group was eliminated was obtained as a main product.

### INDUSTRIAL APPLICABILITY

According to the process of the present invention, indole compounds are obtained in a relatively mild reaction condition even from starting materials having hydrogen reduction sensitive substituents. The indole compounds synthesized according to the process of the present invention are important as fine chemical intermediates for pharmaceuticals and agrochemical, etc., and the availability of the present invention is expected in the future

1. A process for producing an indole compound of formula (2) 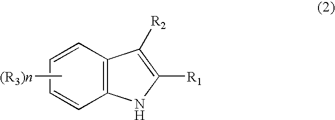
wherein R₁ and R₂ are independently of each other hydrogen atom, an optionally substituted alkyl group, a phenyl group, an alkoxycarbonyl group or an acyl group, R₃ is an optionally substituted alkyl group, a phenyl group, an alkoxy group, a benzyloxy group, an alkoxycarbonyl group, a nitro group or a halogen atom, and n is an integer of 0 to 4, characterized by using carbon monoxide when 2-nitrobenzylcarbony compound of formula (1) 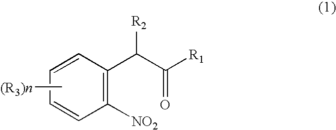
wherein R₁, R₂, R₃ and n have the same meaning as the above, is reduced in the presence of a catalyst comprising a Group VIII metal of the Periodic Table.

2. The process for producing an indole compound according to claim 1, wherein the catalyst comprising a Group VIII metal of the Periodic Table is a metal catalyst selected from an iron catalyst, a ruthenium catalyst, a palladium catalyst, a cobalt catalyst, a rhodium catalyst, a nickel catalyst and a platinum catalyst.

3. The process for producing an indole compound according to claim 1, wherein the catalyst comprising a Group VIII metal of the Periodic Table is a metal catalyst selected from an iron catalyst, a ruthenium catalyst, a palladium catalyst and a platinum catalyst.

4. The process for producing an indole compound according to claim 1, wherein the catalyst comprising a Group VIII metal of the Periodic Table is an iron or ruthenium complex catalyst in which carbon monoxide is coordinated.

5. The process for producing an indole compound according to claim 1, wherein the catalyst comprising a Group VIII metal of the Periodic Table is a palladium catalyst or platinum catalyst in which phosphine type ligand is coordinated.

6. The process for producing an indole compound according to claim 1, wherein R₁ and R₂ are independently of each other hydrogen atom, an optionally substituted alkyl group, an alkoxycarbonyl group or an acyl group, R₃ is an optionally substituted alkyl group or a halogen atom, and n is an integer of 0 to 4.

7. The process for producing an indole compound according to claim 1, wherein R₁ is methyl group, R₂ is hydrogen atom, an alkoxycarbonyl group or an acyl group, R₃ is a halogen atom, and n is an integer of 0 or 1.

8. The process for producing an indole compound according to claim 1, wherein R₁ is methyl group, R₂ is hydrogen atom, R₃ is fluorine atom, and n is an integer of 0 or 1.

9. The process for producing an indole compound according to claim 2, wherein R₁ and R₂ are independently of each other hydrogen atom, an optionally substituted alkyl group, an alkoxycarbonyl group or an acyl group, R₃ is an optionally substituted alkyl group or a halogen atom, and n is an integer of 0 to 4.

10. The process for producing an indole compound according to claim 3, wherein R₁ and R₂ are independently of each other hydrogen atom, an optionally substituted alkyl group, an alkoxycarbonyl group or an acyl group, R₃ is an optionally substituted alkyl group or a halogen atom, and n is an integer of 0 to 4.

11. The process for producing an indole compound according to claim 4, wherein R₁ and R₂ are independently of each other hydrogen atom, an optionally substituted alkyl group, an alkoxycarbonyl group or an acyl group, R₃ is an optionally substituted alkyl group or a halogen atom, and n is an integer of 0 to 4.

12. The process for producing an indole compound according to claim 5, wherein R₁ and R₂ are independently of each other hydrogen atom, an optionally substituted alkyl group, an alkoxycarbonyl group or an acyl group, R₃ is an optionally substituted alkyl group or a halogen atom, and n is an integer of 0 to 4.

13. The process for producing an indole compound according to claim 2, wherein R₁ is methyl group, R₂ is hydrogen atom, an alkoxycarbonyl group or an acyl group, R₃ is a halogen atom, and n is an integer of 0 or 1.

14. The process for producing an indole compound according to claim 3, wherein R₁ is methyl group, R₂ is hydrogen atom, an alkoxycarbonyl group or an acyl group, R₃ is a halogen atom, and n is an integer of 0 or 1.

15. The process for producing an indole compound according to claim 4, wherein R₁ is methyl group, R₂ is hydrogen atom, an alkoxycarbonyl group or an acyl group, R₃ is a halogen atom, and n is an integer of 0 or 1.

16. The process for producing an indole compound according to claim 5, wherein R₁ is methyl group, R₂ is hydrogen atom, an alkoxycarbonyl group or an acyl group, R₃ is a halogen atom, and n is an integer of 0 or 1.

17. The process for producing an indole compound according to claim 2, wherein R₁ is methyl group, R₂ is hydrogen atom, R₃ is fluorine atom, and n is an integer of 0 or 1.

18. The process for producing an indole compound according to claim 3, wherein R₁ is methyl group, R₂ is hydrogen atom, R₃ is fluorine atom, and n is an integer of 0 or 1.

19. The process for producing an indole compound according to claim 4, wherein R₁ is methyl group, R₂ is hydrogen atom, R₃ is fluorine atom, and n is an integer of 0 or 1.

20. The process for producing an indole compound according to claim 5, wherein R₁ is methyl group, R₂ is hydrogen atom, R₃ is fluorine atom, and n is an integer of 0 or 1.
